(19) United States
(12) Patent Application Publication
(10) Pub. No.: US 2026/0294282 A1
(43) Pub. Date: Oct. 1, 2026

(54) PORTABLE IDENTIFICATION DEVICE

(71) Applicant: ATomic Wrks L.L.C., Rocky Mount, NC (US)

(72) Inventor: Diego Nicolas Roggero, Nashville, NC (US)

(21) Appl. No.: 19/092,543

(22) Filed: Mar. 27, 2025

(51) Int. Cl. A61B 5/1172; B26B 11/00

(52) U.S. Cl. CPC A61B 5/1172; B26B 11/00

(57) ABSTRACT

A portable identification device for the capture of personal identification information. The portable identification device enables the capture of latent fingerprints. The portable identification device includes a body, a cutting component, and a covering component. The portable identification device can receive and modify identification cards for the capture of personal identification information.

### BACKGROUND

Many law enforcement officers and related public service positions must capture personal identification information (e.g., latent fingerprints) when carrying out their duties. This is a critical component of these professions because the captured personal identification information may be used as evidence. However, capturing the personal identification information can be difficult due to the lack of portable equipment for the capture of fingerprints.

### TECHNICAL FIELD

The present disclosure is generally directed to a portable identification device, more specifically to a portable identification device for capturing fingerprints.

### DESCRIPTION OF RELATED ART

Current personal identification devices include digital live scanning equipment. However, these devices cannot be used in many conditions that arise during a law enforcement officer's shift. Therefore, there is a need for a portable identification device that improves the capture of fingerprints.

### BRIEF SUMMARY

In some aspects of the present disclosure, a portable identification device is disclosed. The portable identification device may include a body, a tape bar, a pivot bar, a retention bar, a hexagon blade face, and a circular blade face. The portable identification device can hold tape, include a folding blade holder, a finger groove for improving removal of fingerprint cards, a name plate for serialization, a grip (e.g., rubber feet), and/or wire retention for a key ring to maintain positioning of a tape bar. The portable identification device can capture fingerprints. A fingerprint includes a legible circle (center of the loop) and triangles (deltas).

In at least one embodiment of the present disclosure, a portable identification device is disclosed. The portable identification device can include a body, at least one cutting component, and at least one covering component. The body can receive an identification card. The at least one covering component can distribute a layer of material over the identification card. The at least one covering component is designed to cut the layer of material and/or the identification card.

In at least one aspect of the present disclosure, the at least one covering component is pivotably connected to the body. In at least one aspect, the at least one cutting component is pivotably attached to the body. In at least one aspect of the present disclosure, the portable identification device further comprises an identification component. The identification component can include a stamping tool that is operable to stamp the identification card. In at least one aspect of the present disclosure, the portable identification device further includes a dust container and/or a dusting wand. In at least one aspect of the present disclosure, the portable identification device includes at least two grip components. The at least two grip components include at least one grip component on a first end of the body and at least one grip component on a second end of the body. In at least one aspect of the present disclosure, the portable identification device includes a pivot bar and a retention bar. The pivot bar enables storage of the at least one cutting component and the retention bar supports the at least one cutting component. In at least one aspect of the present disclosure, the body of the portable identification device includes a top portion and a bottom portion. The top portion is removably connected to the bottom portion. In at least one aspect of the present disclosure, the body includes a slot to receive an identification card. In at least one aspect of the present disclosure, the at least one cutting component is positioned to run along an end of the slot.

In at least one embodiment of the present disclosure, a portable identification device is disclosed. The portable identification device includes a body, at least one cutting component, and at least one covering component. The body is operable to receive an identification card. The at least one cutting component and the at least one covering component are pivotably connected to the body. The at least one covering component is operable to distribute a layer of material over the identification card. The at least one cutting component is designed to cut the layer of material and/or the identification card.

In at least one aspect of the present disclosure, the portable identification device includes a body including a top portion and a bottom portion. The top portion is removably connected to the bottom portion. In at least one embodiment of the present disclosure, the body of the portable identification device includes a slot to receive the identification card. In at least one aspect of the present disclosure, the at least one cutting component is positioned to run along an end of the slot. In at least one aspect of the present disclosure, the portable identification device includes an identification component. The identification component includes a stamping tool that is operable to stamp the identification card. In at least one aspect of the present disclosure, the portable identification device can include a dust container and/or a dusting wand. In at least one aspect of the present disclosure, the portable identification device includes at least two grip components. The at least two grip components include at least one grip component positioned on a first end of the body and at least one grip component positioned on a second end of the body. In at least one aspect of the present disclosure, the portable identification device includes a pivot bar and a retention bar. The pivot bar enables storage of the at least one cutting component. The retention bar supports the at least one cutting component.

In at least one aspect of the present disclosure, a portable identification device is disclosed. The portable identification device includes a body, at least one cutting assembly including a cutting component and a pivot component, and at least one covering component. The body can receive an identification card via at least one slot. The at least one cutting component and the at least one covering component are pivotably connected to the body. Via the pivot component, the at least one cutting component can move between a stored position and an open position. The at least one covering component can distribute a layer of material over the identification card. The at least one cutting component can cut the layer of material and/or the identification card. In at least one aspect of the present disclosure, the at least one cutting component can run along an end of the at least one slot.

### BRIEF DESCRIPTION OF THE SEVERAL VIEWS OF THE DRAWINGS

The embodiments illustrated, described, and discussed herein are illustrative of the present disclosure. As these embodiments of the present disclosure are described with reference to illustrations, various modifications or adaptations of the methods and or specific structures described may become apparent to those skilled in the art. It will be appreciated that modifications and variations are covered by the above teachings and within the scope of the appended claims without departing from the spirit and intended scope thereof. All such modifications, adaptations, or variations that rely upon the teachings of the present disclosure, and through which these teachings have advanced the art, are considered to be within the spirit and scope of the present disclosure. Hence, these descriptions and drawings should not be considered in a limiting sense, as it is understood that the present disclosure is in no way limited to only the embodiments illustrated.

FIG. 1 illustrates a front view of a portable identification device according to an embodiment of the present disclosure.

FIG. 2 illustrates an exploded front view of a portable identification device according to an embodiment of the present disclosure.

FIG. 3 illustrates an exploded side perspective view of a portable identification device according to an embodiment of the present disclosure.

FIG. 4 illustrates an exploded rear perspective view of a portable identification device according to an embodiment of the present disclosure.

FIG. 5 illustrates an exploded rear view of a portable identification device according to an embodiment of the present disclosure.

FIG. 6 illustrates an exploded side perspective view of a portable identification device according to an embodiment of the present disclosure.

FIG. 7 illustrates a front exploded view of a portable identification device according to an embodiment of the present disclosure.

FIG. 8 illustrates an exploded front perspective view of a portable identification device according to an embodiment of the present disclosure.

FIG. 9 illustrates an exploded front perspective view of a portable identification device according to an embodiment of the present disclosure.

FIG. 10 illustrates an exploded front perspective view of a portable identification device according to an embodiment of the present disclosure.

FIG. 11 illustrates an exploded front perspective view of a portable identification device according to an embodiment of the present disclosure.

FIG. 12 illustrates a rear exploded view of a portable identification device according to an embodiment of the present disclosure.

FIG. 13 illustrates a perspective view of a portable identification device according to an embodiment of the present disclosure.

FIG. 14 illustrates a top perspective view of a portable identification device according to an embodiment of the present disclosure.

FIG. 15 illustrates a top perspective view of a portable identification device according to an embodiment of the present disclosure.

FIG. 16 illustrates a front view of an assembled portable identification device according to an embodiment of the present disclosure.

FIG. 17 illustrates a side view of a portable identification device according to an embodiment of the present disclosure.

FIG. 18 illustrates a rear view of a portable identification device according to an embodiment of the present disclosure.

FIG. 19 illustrates a rear view of a portable identification device according to an embodiment of the present disclosure.

FIG. 20 illustrates a side view of a portable identification device according to an embodiment of the present disclosure.

FIG. 21 illustrates a front view of a portable identification device according to an embodiment of the present disclosure.

FIG. 22 illustrates a side view of a portable identification device according to an embodiment of the present disclosure.

FIG. 23 illustrates a side view of a portable identification device according to an embodiment of the present disclosure.

FIG. 24 illustrates a front perspective view of a portable identification device according to an embodiment of the present disclosure.

FIG. 25 illustrates a rear perspective view of a portable identification device according to an embodiment of the present disclosure.

FIG. 26 illustrates a rear perspective view of a portable identification device according to an embodiment of the present disclosure.

FIG. 27 illustrates a perspective view of a portion of a portable identification device according to an embodiment of the present disclosure.

FIG. 28 illustrates a perspective view of a portion of a portable identification device according to an embodiment of the present disclosure.

FIG. 29 illustrates a perspective view of a portion of a portable identification device according to an embodiment of the present disclosure.

FIG. 30 illustrates a perspective view of a portion of a portable identification device according to an embodiment of the present disclosure.

FIG. 31 illustrates a top perspective view of a portion of a portable identification device according to an embodiment of the present disclosure.

FIG. 32A illustrates a top view of a portable identification device according to an embodiment of the present disclosure.

FIG. 32B illustrates a top view of a portable identification device according to an embodiment of the present disclosure.

FIG. 33 illustrates a perspective view of a portable identification device according to an embodiment of the present disclosure.

FIG. 34 illustrates a blade assembly of a portable identification device according to an embodiment of the present disclosure.

FIG. 35 illustrates a blade assembly of a portable identification device according to an embodiment of the present disclosure.

FIG. 36 illustrates an exploded view of an interior of a portable identification device according to an embodiment of the present disclosure.

FIG. 37 illustrates an exploded view of an interior of a portable identification device according to an embodiment of the present disclosure.

### DETAILED DESCRIPTION

For the purposes of promoting an understanding of the present disclosure, reference will be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e., at least one) of the grammatical object of the article. By way of example, “a composite” means at least one composite and can include more than one composite.

Throughout the specification, the terms “about” and/or “approximately” may be used in conjunction with numerical values and/or ranges. The term “about” is understood to mean those values near to a recited value. For example, “about 40 [units]” ay mean within +/−25% of 40 (e.g., from 30 to 50), within +/−20%, +/−15%, +/−10%, +/−9%, +/−8%, +/−7%, +/−6%, +/−5%, +/−4%, +/−3%, +/−2%, +/−1%, less than +/−1%, or any other value or range of values therein or there below. Furthermore, the phrases “less than about [a value]” or “greater than about [a value]” should be understood in view of the definition of the term “about” provided herein. The terms “about” and “approximately” may be used interchangeably.

As used herein, the verb “comprise” as is used in this description and in the claims and its conjugations are used in its non-limiting sense to mean that items following the word are included, but items not specifically mentioned are not excluded.

Throughout the specification the word “comprising,” or variations such as “comprises” or “comprising,” will be understood to imply the inclusion of a stated element, integer or step, or group of elements, integers, or steps, but not the exclusion of any other element, integer or step, or group of elements, integers or steps. The present disclosure may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only”, and the like in connection with the recitation of claim elements, or the use of a “negative” limitation.

Unless defined otherwise, all technical and scientific terms used herein have the same meanings as commonly understood by one of ordinary skill in the art to which this disclosure belongs. Preferred methods, devices, and materials are described, although any methods and materials similar or equivalent to those described herein can be used in the practice or testing of the present disclosure. All references cited herein are incorporated by reference in their entirety.

In an aspect of the present disclosure, a portable identification device is disclosed. The portable identification device is designed for the capture of fingerprints. The portable identification device includes a body, a taper bar, a pivot bar, a retention bar, a hexagon blade face, and/or a circular blade face. The portable identification device can (1) include fingerprint tape, (2) a folding blade and corresponding holder for improving storage, (3) a finger groove for improving the removal of identification cards (e.g., fingerprints), (4) a serialization name plate, (5) a grip component (e.g., rubber feet), and/or (6) a wire retention component.

In another aspect of the present disclosure, a method for capturing a fingerprint (e.g., latent, elimination) is disclosed. The method may include (1) placing at least one finger of an individual on an ink container of a portable identification device, (2) placing the inked finger on a body of the portable identification device, (3) removing the inked finger, leaving a fingerprint on a identification card, (4) running fingerprint tape over the identification card, (5) cutting, using a cutting component (e.g., a blade), the identification card to a desired size, (6) removing the taped identification card, and (7) storing the taped identification card in a storage component of the portable fingerprint device.

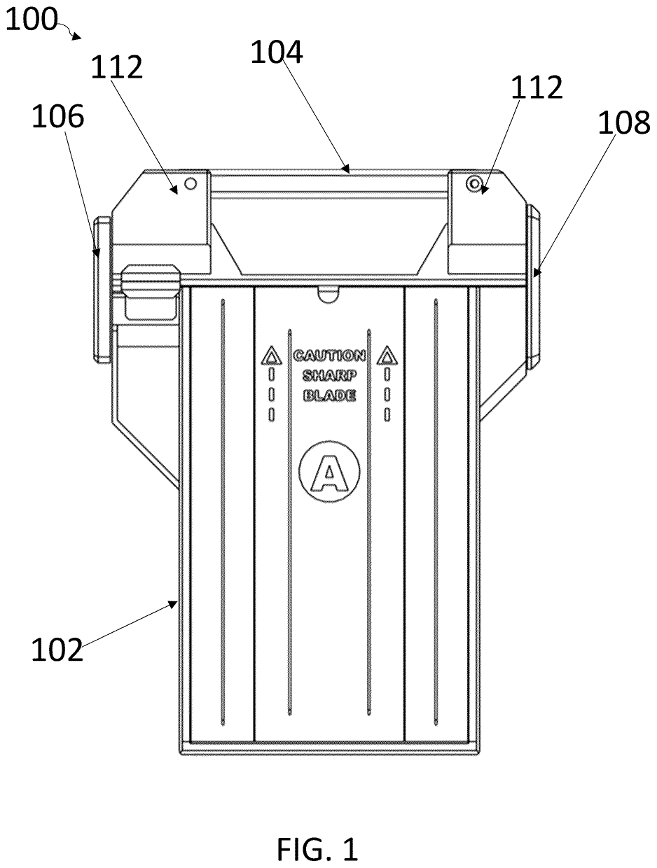

FIG. 1 illustrates a front view of a portable identification device according to an embodiment of the present disclosure. The portable identification device 100 includes a body 102, a tape bar 104, a pivot bar 106, a retention bar 108, a hexagon blade face, a circular blade face, and a plurality of grip components 110. The portable identification device 100 is operable to receive an identification card via the body 102. The identification card can be a piece of paper, a fingerprint card, and/or other materials for capturing a fingerprint.

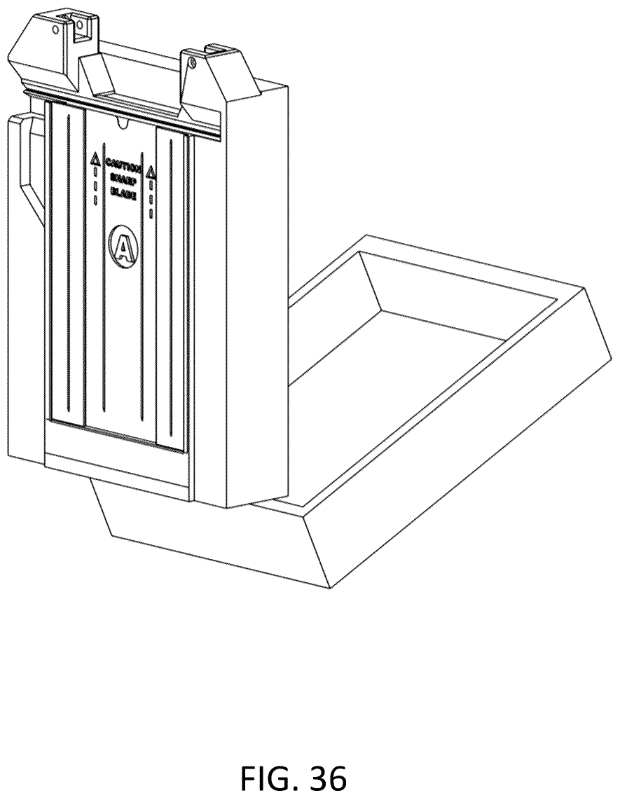

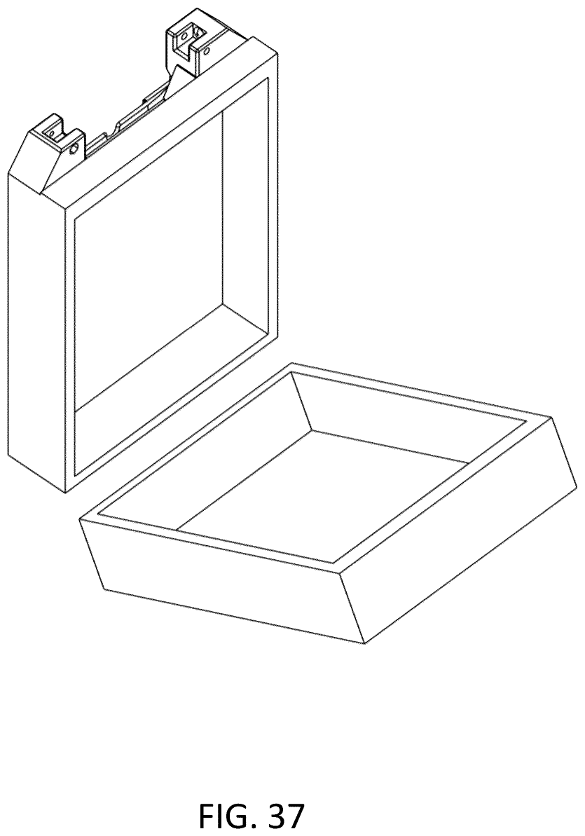

Once the identification card is positioned on the body of the portable identification device, a user can press their fingers onto the identification card. The portable identification card may include an ink/dust container and/or a dusting wand for capturing the fingerprint. Once the fingerprint is captured, fingerprint tape can be rolled over the identification card. The fingerprint tape may be positioned on the tape bar 104 and is designed to unroll around the tape bar. Once the fingerprint tape is positioned over the identification card, a cutting component (e.g., blade) of the portable identification device can cut the identification card and/or the fingerprint tape. Once the tape and/or identification card is cut, the taped identification card can be removed from the portable identification device. In some embodiments, the portable identification device may include a storage component for storing the taped identification card. For example, and without limitation, as shown in FIGS. 36 and 37, the body of the portable identification device may include a top portion and a bottom portion that is removably connected. The top portion can rotate and/or pivot away from the bottom portion to reveal an interior storage portion. The plurality of grip components 112 can be designed to receive one or more fingers to improve the handling of the portable identification device.

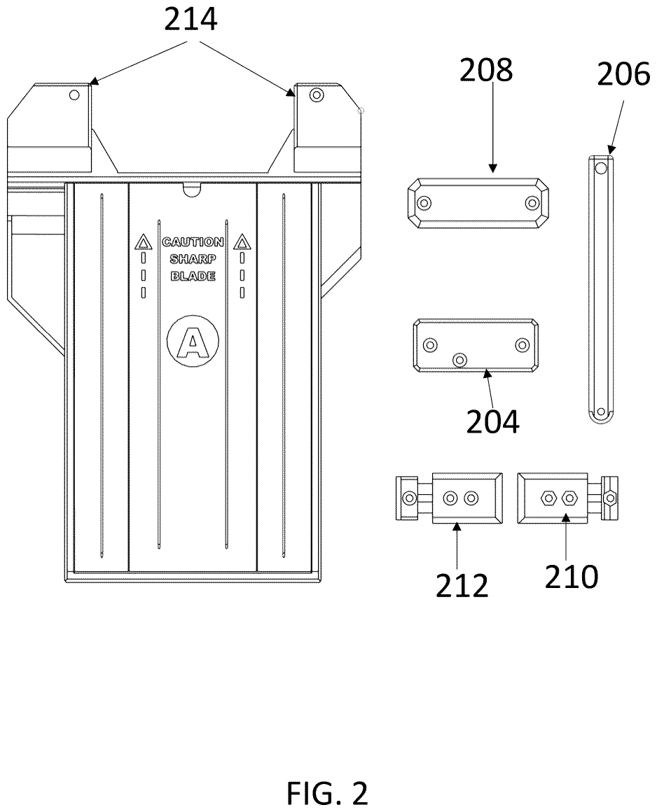

FIG. 2 illustrates a front perspective view of a portable identification device according to an embodiment of the present disclosure. The portable identification device 200 includes a body 202, a pivot bar 204, a tape bar 206, a retention bar 208, a hexagon blade face 210, and a circular blade face 212. The portable identification device can include a grip component 214 design to improve grip by a user. The pivot bar 204 can enable a blade assembly to fold into and out of the body of the portable identification device. The tape bar 206 is pivotably connected to the body 202 to removal of the fingerprint tape. The retention bar prevents a razor blade from falling out of the portable identification device. The retention bar may be removably connected to the body. Advantageously, having a removably connected retention bar improves vibration and force distribution when the portable identification device is dropped. The hexagonal blade face and the circular blade face can work in combination to hold the razor blade in place.

In at least one embodiment of the present disclosure, the portable identification device includes an identification component. The identification component can be utilized to label, mark, and/or similarly identify an identification card. For example, and without limitation, the identification component can include a metal stamping component (e.g., dog tag). Marking the plastic identification card can be inefficient, therefore using a metal stamping component can properly mark an identification card without affecting the identification.

In at least one embodiment of the present disclosure, the portable identification device can include an ink/dust container and/or a dusting wand. In some embodiments, the ink/dust container and/or the dusting wand may be integrated into the body of the portable identification device. In some embodiments the ink/dust container and/or the dusting wand may be removably connected to the body.

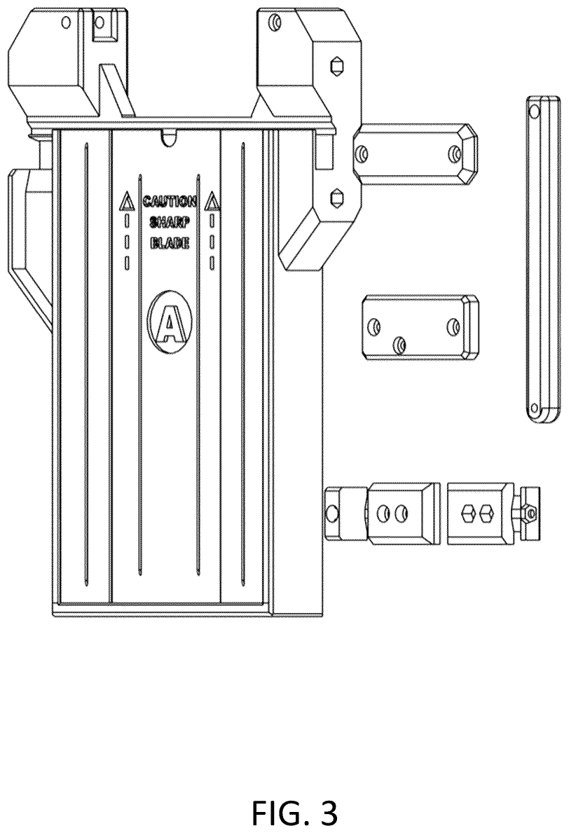

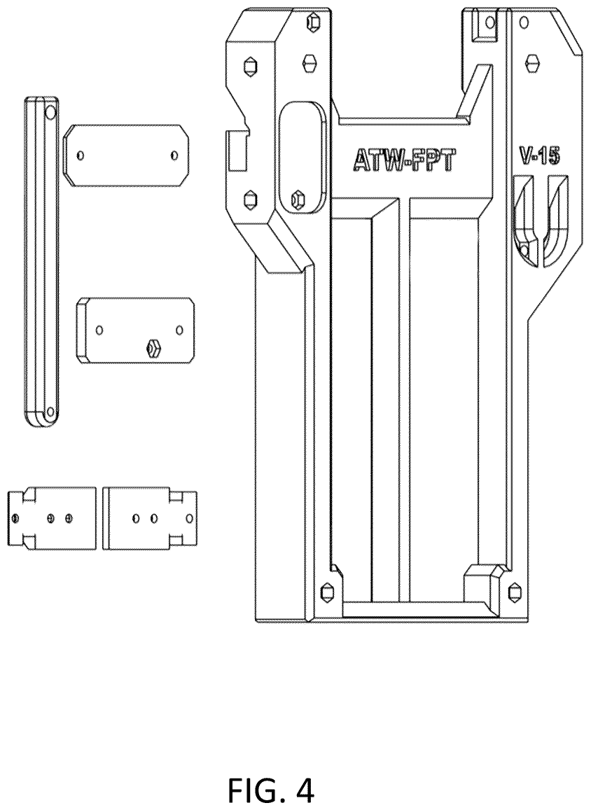

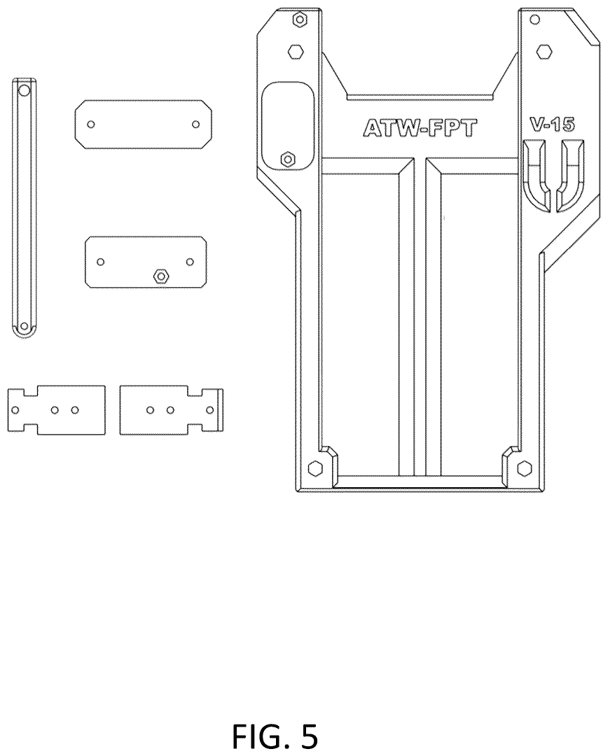

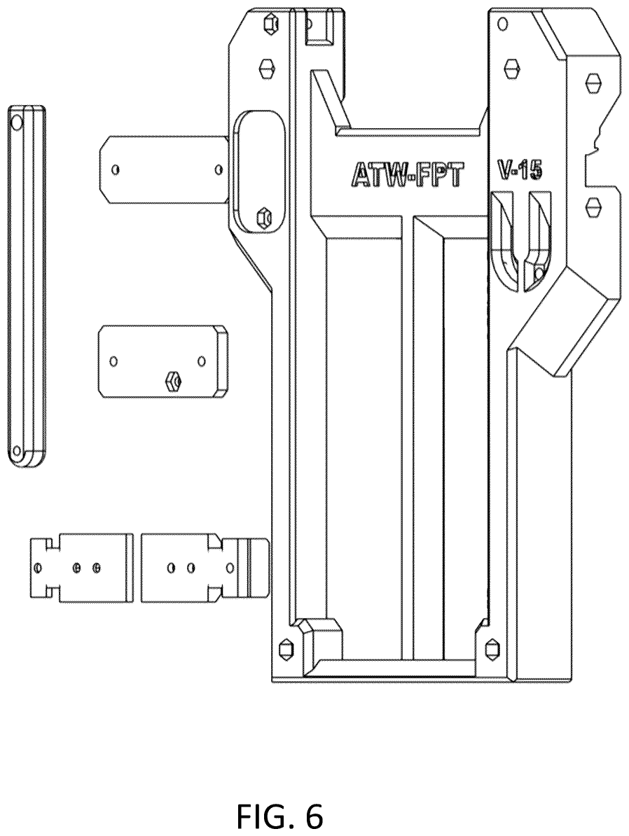

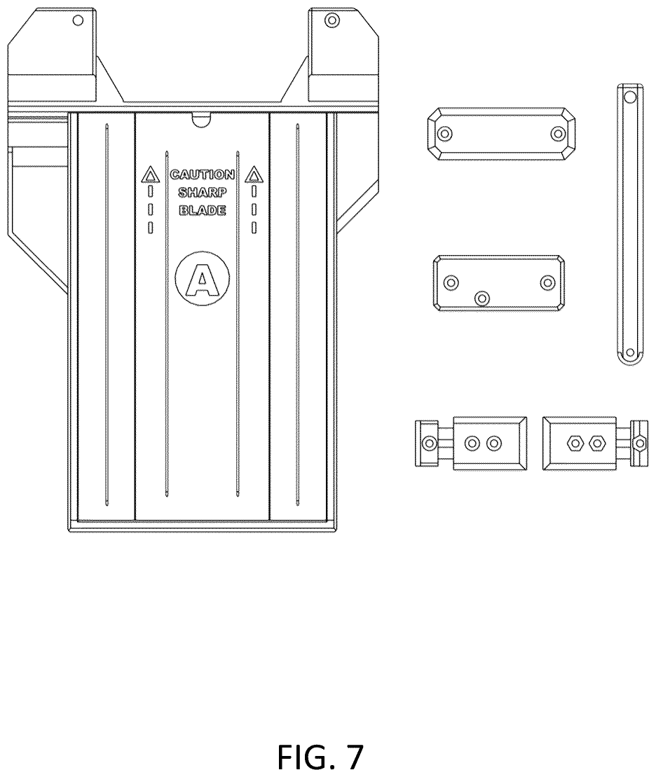

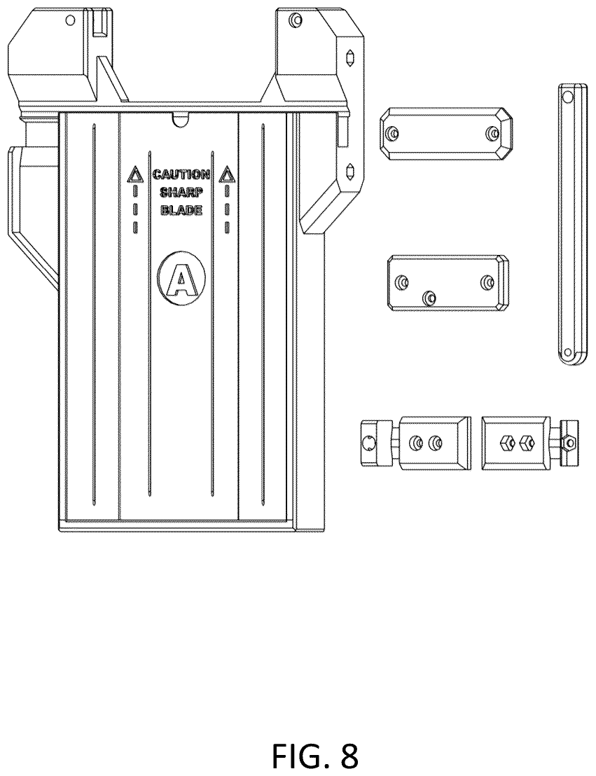

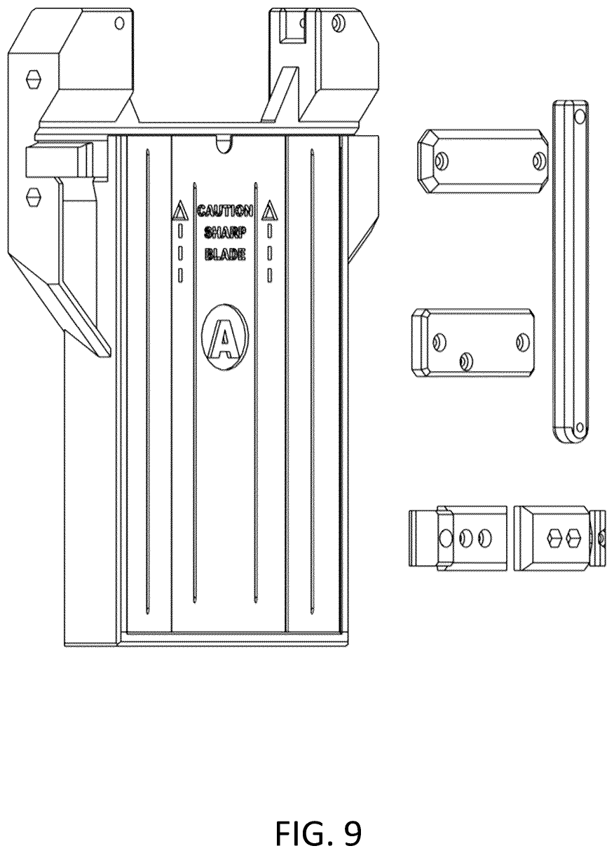

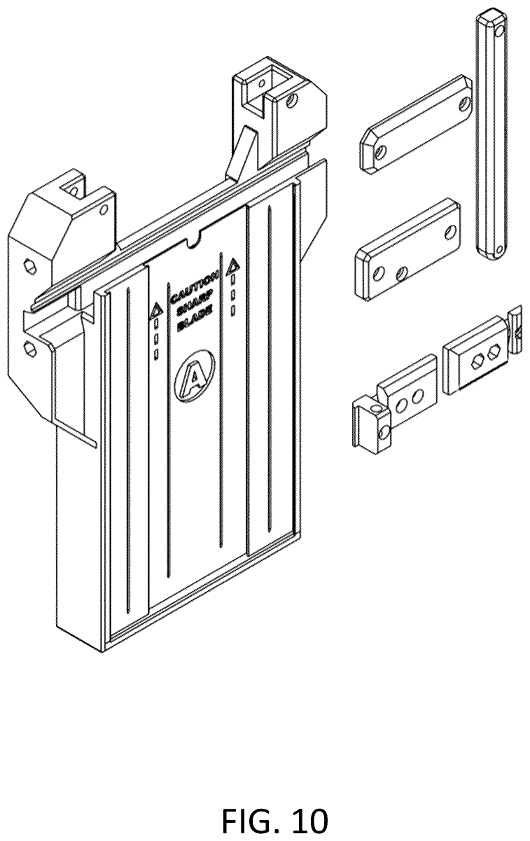

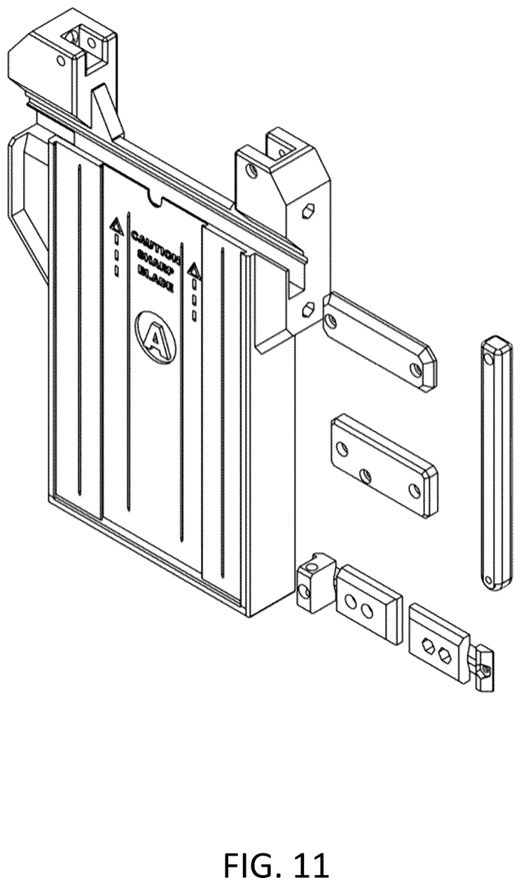

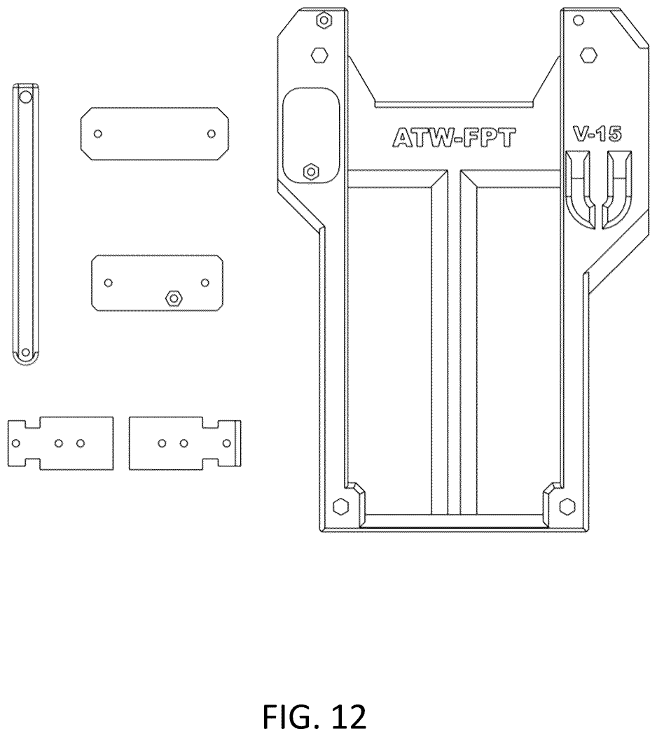

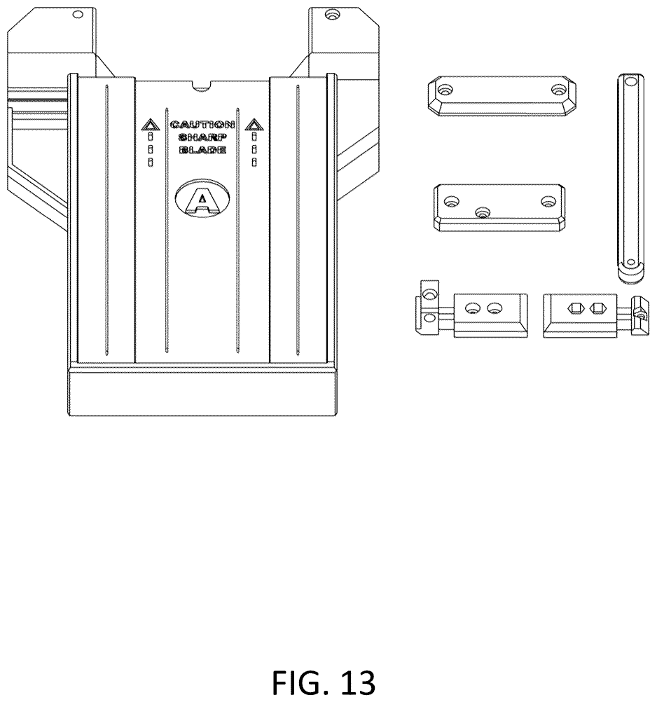

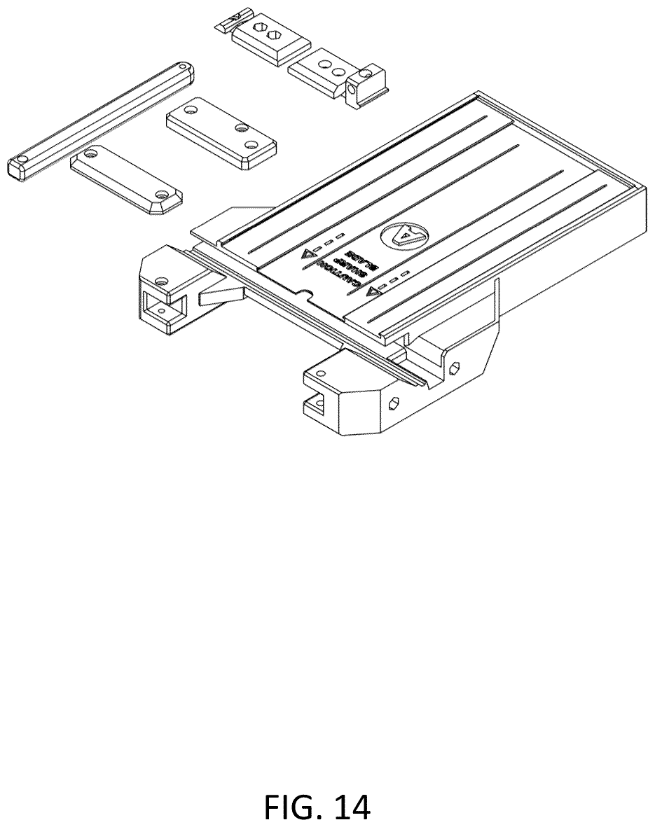

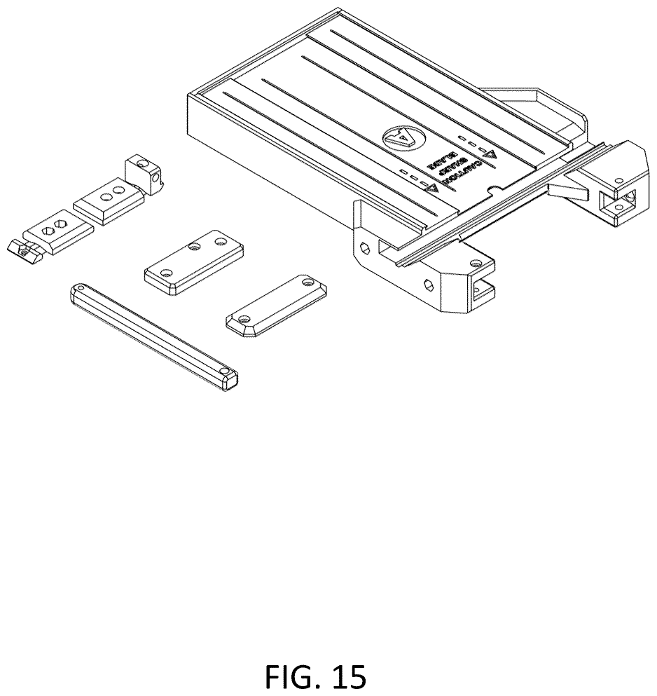

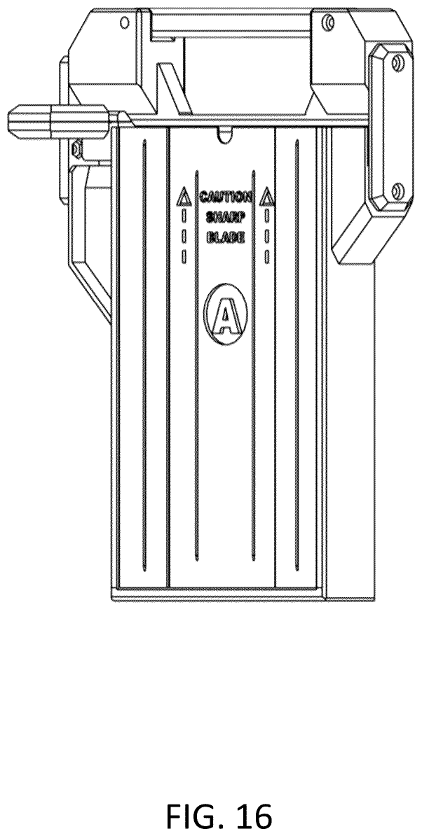

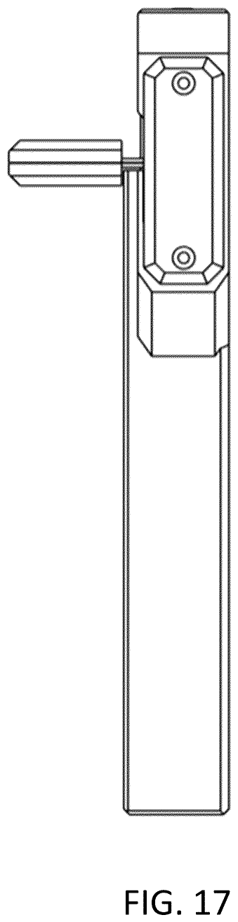

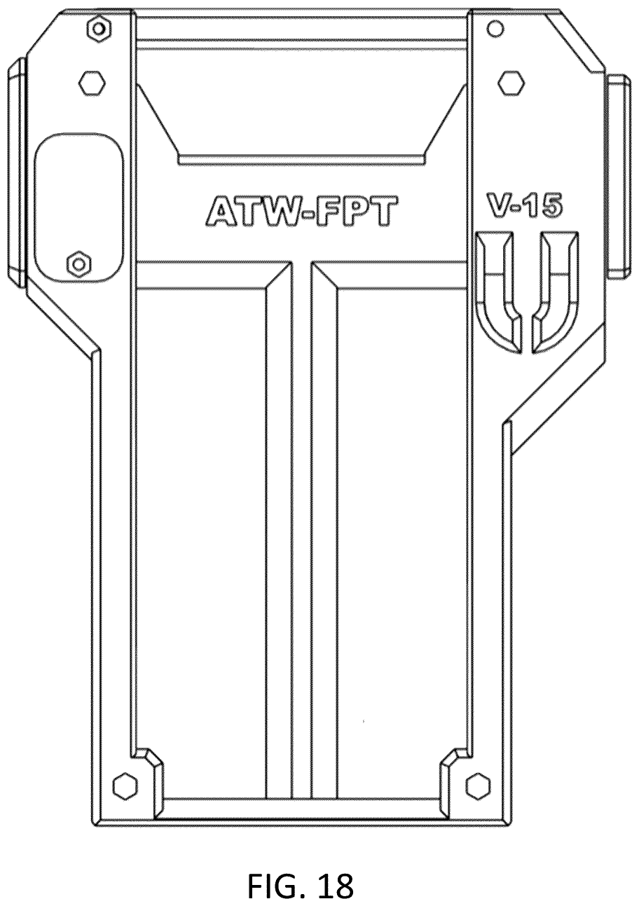

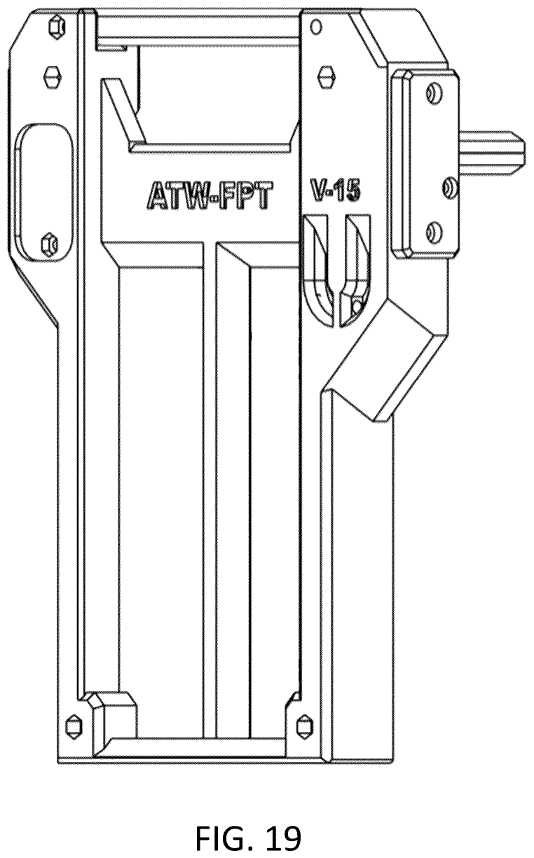

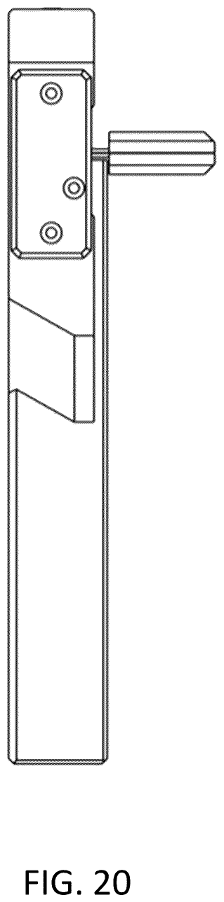

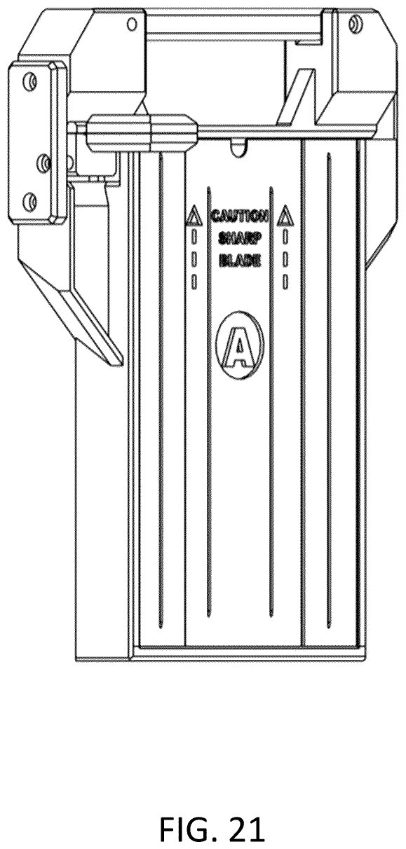

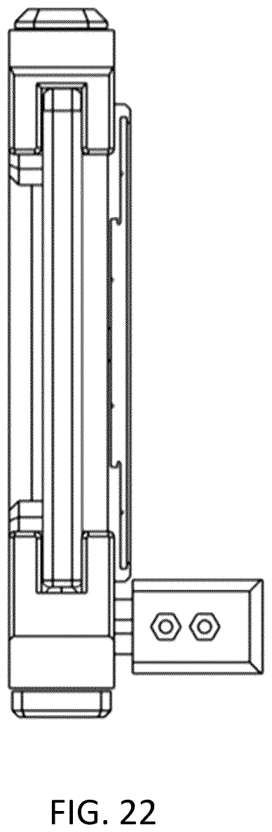

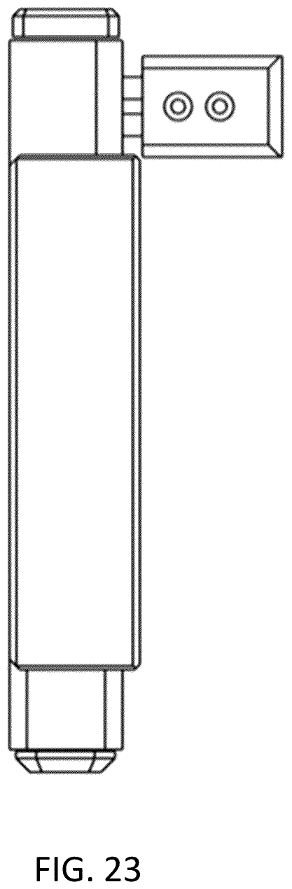

FIG. 3 illustrates an exploded side perspective view of a portable identification device according to an embodiment of the present disclosure. FIG. 4 illustrates an exploded rear perspective view of a portable identification device according to an embodiment of the present disclosure. FIG. 5 illustrates an exploded rear view of a portable identification device according to an embodiment of the present disclosure. FIG. 6 illustrates an exploded side perspective view of a portable identification device according to an embodiment of the present disclosure. FIG. 7 illustrates a front exploded view of a portable identification device according to an embodiment of the present disclosure. FIG. 8 illustrates an exploded front perspective view of a portable identification device according to an embodiment of the present disclosure. FIG. 9 illustrates an exploded front perspective view of a portable identification device according to an embodiment of the present disclosure. FIG. 10 illustrates an exploded front perspective view of a portable identification device according to an embodiment of the present disclosure. FIG. 11 illustrates an exploded front perspective view of a portable identification device according to an embodiment of the present disclosure. FIG. 12 illustrates a rear exploded view of a portable identification device according to an embodiment of the present disclosure. FIG. 13 illustrates a perspective view of a portable identification device according to an embodiment of the present disclosure. FIG. 14 illustrates a top perspective view of a portable identification device according to an embodiment of the present disclosure. FIG. 15 illustrates a top side perspective view of a portable identification device according to an embodiment of the present disclosure. FIG. 16 illustrates a front view of an assembled portable identification device according to an embodiment of the present disclosure. FIG. 17 illustrates a side view of a portable identification device according to an embodiment of the present disclosure. FIG. 18 illustrates a rear view of a portable identification device according to an embodiment of the present disclosure. FIG. 19 illustrates a rear view of a portable identification device according to an embodiment of the present disclosure. FIG. 20 illustrates a side view of a portable identification device according to an embodiment of the present disclosure. FIG. 21 illustrates a front view of a portable identification device according to an embodiment of the present disclosure. FIG. 22 illustrates a side view of a portable identification device according to an embodiment of the present disclosure. FIG. 23 illustrates a side view of a portable identification device according to an embodiment of the present disclosure.

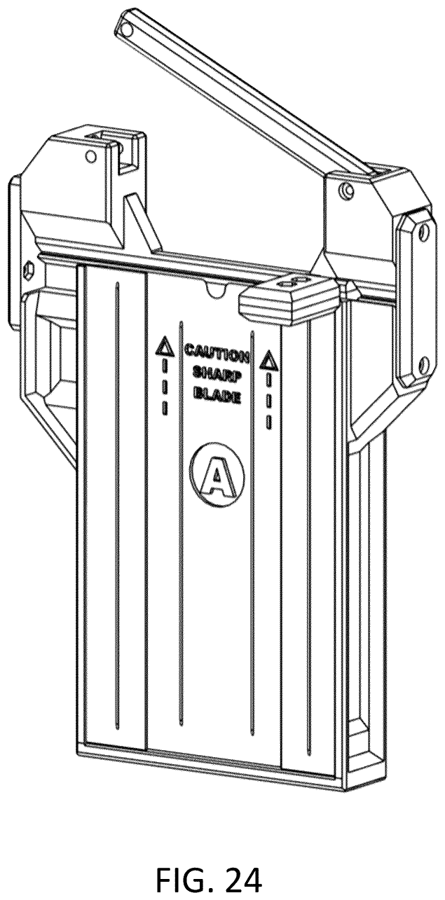

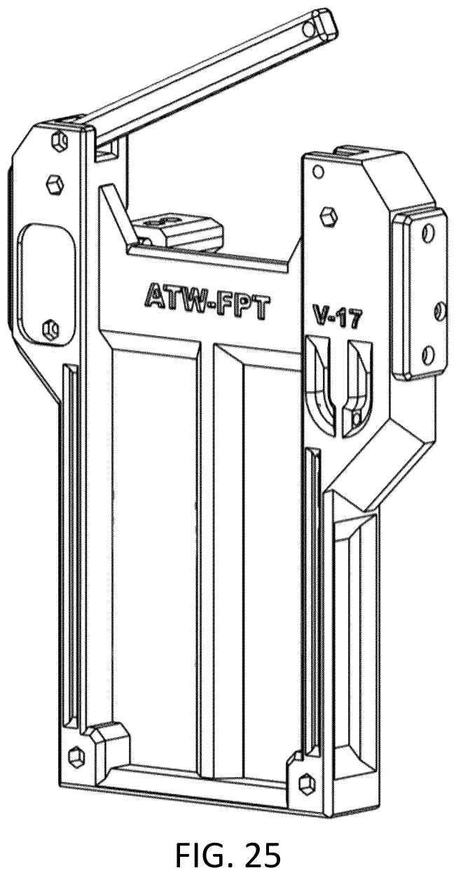

FIG. 24 illustrates a front perspective view of a portable identification device according to an embodiment of the present disclosure. FIG. 25 illustrates a rear perspective view of a portable identification device according to an embodiment of the present disclosure.

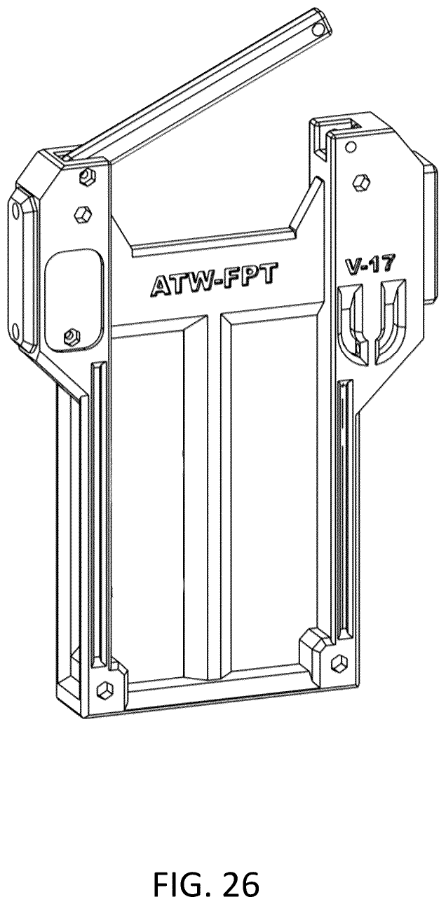

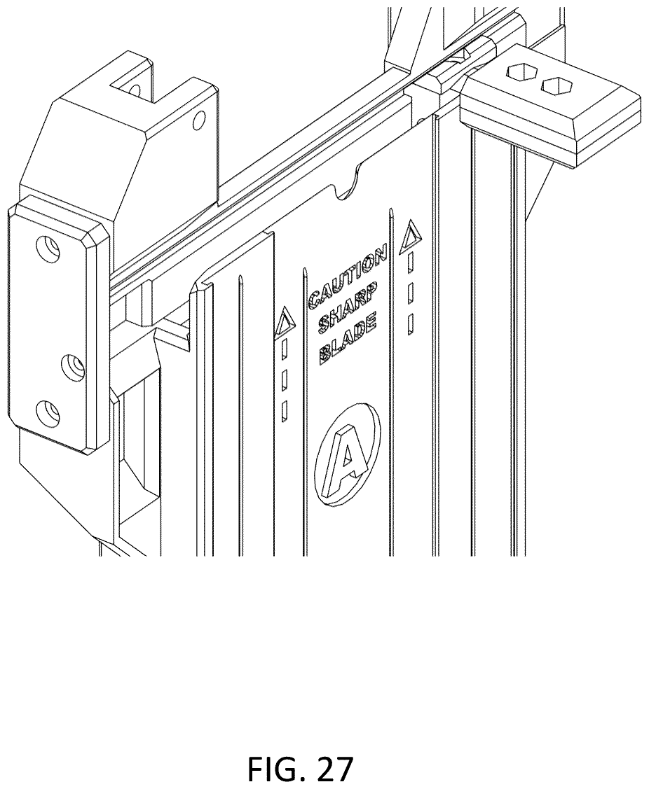

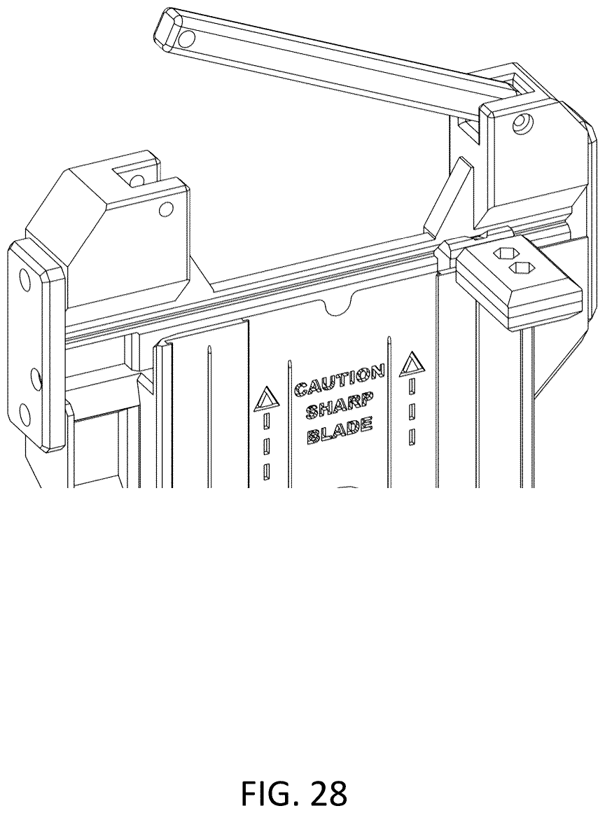

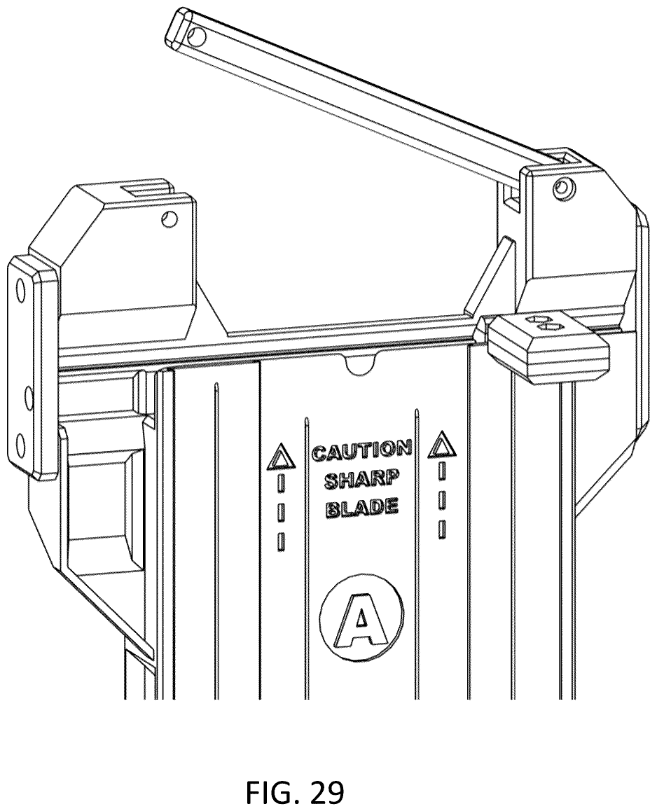

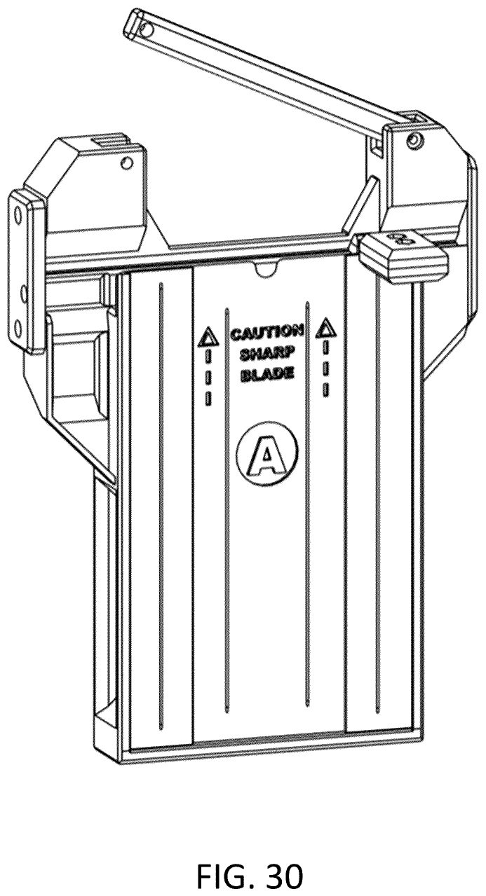

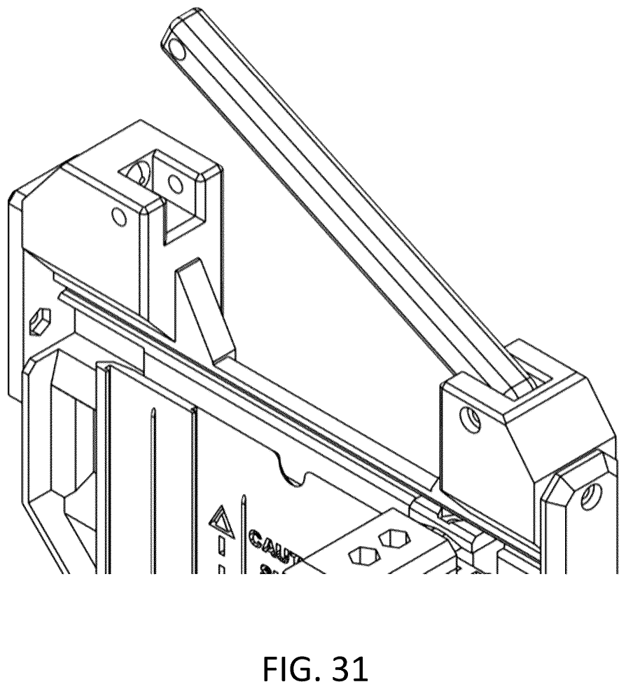

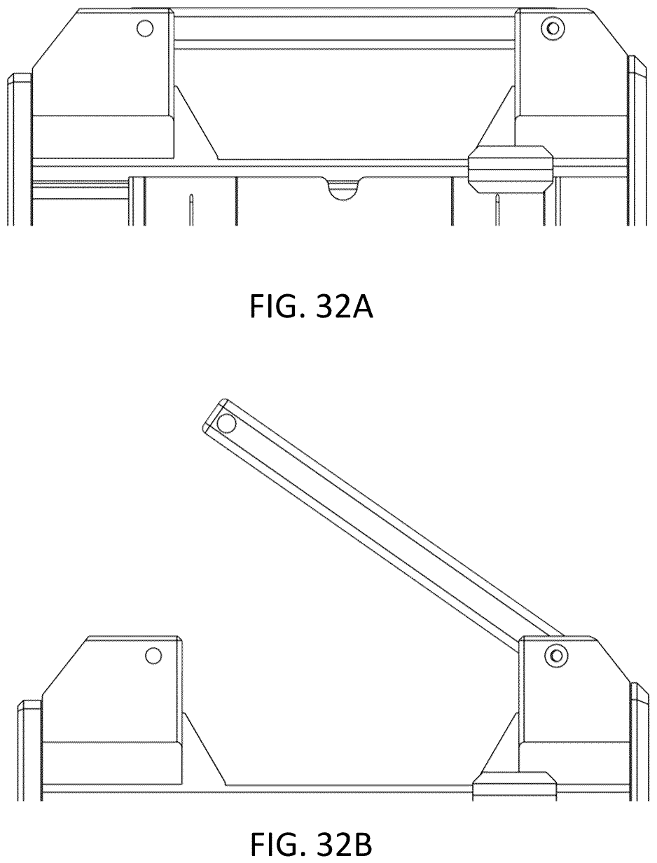

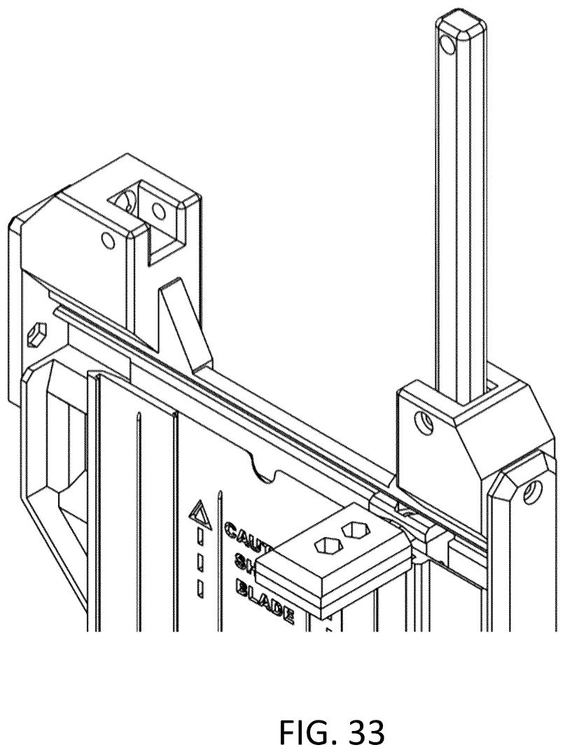

FIG. 26 illustrates a rear perspective view of a portable identification device according to an embodiment of the present disclosure. FIG. 27 illustrates a perspective view of a portion of a portable identification device according to an embodiment of the present disclosure. FIG. 28 illustrates a perspective view of a portion of a portable identification device according to an embodiment of the present disclosure. FIG. 29 illustrates a perspective view of a portion of a portable identification device according to an embodiment of the present disclosure. FIG. 30 illustrates a perspective view of a portion of a portable identification device according to an embodiment of the present disclosure. FIG. 31 illustrates a top perspective view of a portion of a portable identification device according to an embodiment of the present disclosure. FIG. 32A illustrates a top view of a portable identification device according to an embodiment of the present disclosure. FIG. 32B illustrates a top view of a portable identification device according to an embodiment of the present disclosure. FIG. 33 illustrates a perspective view of a portable identification device according to an embodiment of the present disclosure.

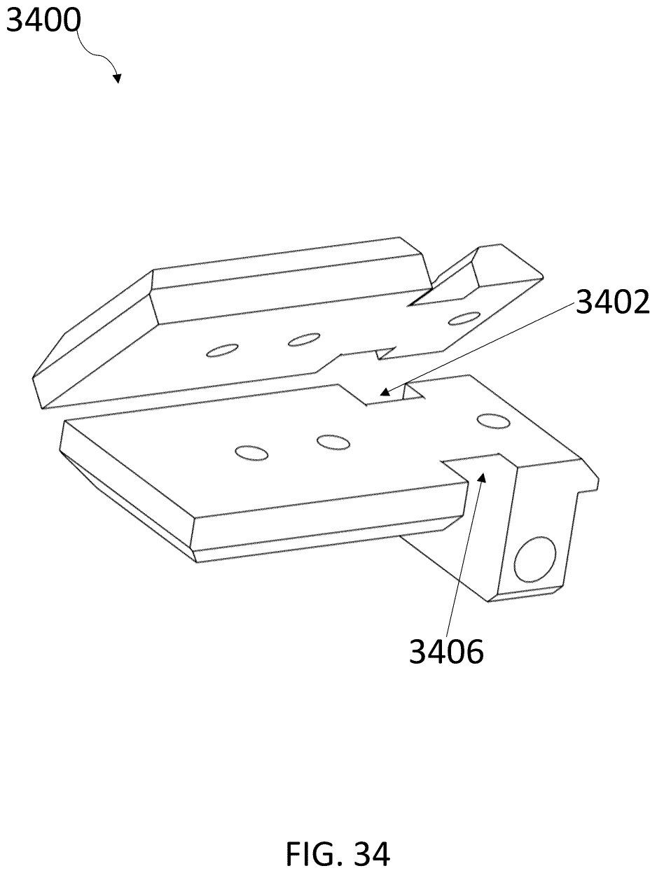

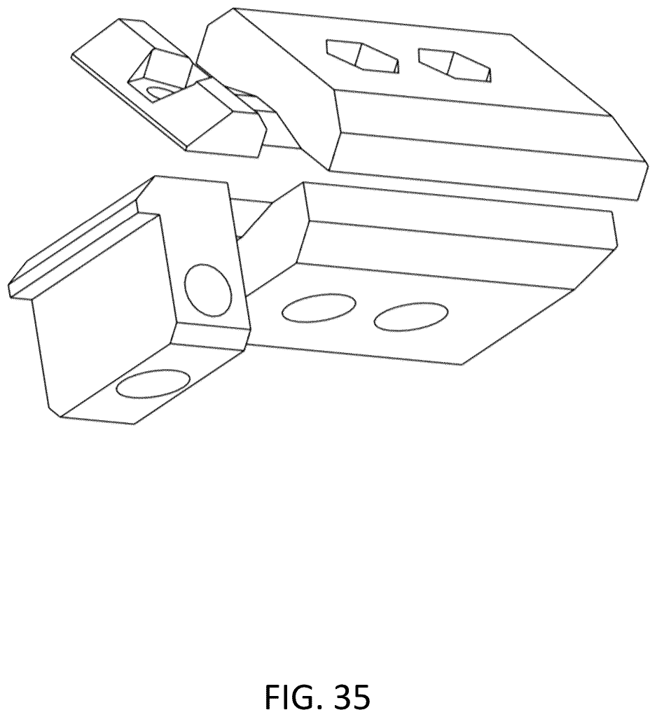

FIG. 34 illustrates a blade assembly of a portable identification device according to an embodiment of the present disclosure. The blade assembly 3400 can receive a cutting component (e.g., blade) in slot 3402 and 3406. FIG. 35 illustrates a blade assembly of a portable identification device according to an embodiment of the present disclosure.

FIG. 36 illustrates an exploded view of an interior of a portable identification device according to an embodiment of the present disclosure. FIG. 37 illustrates an exploded view of an interior of a portable identification device according to an embodiment of the present disclosure.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description but is not intended to be exhaustive or limited to the embodiments in the form disclosed. Many modifications and variations will be apparent to those of ordinary skill in the art without departing from the scope and spirit of the present disclosure. The embodiment was chosen and described in order to best explain the principles of the present disclosure and the practical application, and to enable others of ordinary skill in the art to understand the present disclosure for various embodiments with various modifications as are suited to the particular use contemplated.

The descriptions of the various embodiments of the present disclosure have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A portable identification device comprising:
a body;
at least one cutting component; and
at least one covering component;
wherein the body is operable to receive an identification card;
wherein the at least one covering component is operable to distribute a layer of material over the identification card; and
wherein the at least one cutting component is designed to cut the layer of material and/or the identification card.

2. The portable identification device of claim 1, wherein the at least one covering component is pivotably connected to the body.

3. The portable identification device of claim 1, wherein the at least one cutting component is pivotably attached to the body.

4. The portable identification device of claim 1 further comprising an identification component, wherein the identification component includes a stamping tool, wherein the stamping tool is operable to stamp the identification card.

5. The portable identification device of claim 1 further including a dust container and/or a dusting wand.

6. The portable identification device of claim 1 further comprising at least two grip components, wherein at least one grip component is positioned on a first end of the body, wherein at least one grip component is positioned on a second end of the body.

7. The portable identification device of claim 1 further comprising a pivot bar and a retention bar, wherein the pivot bar enables storage of the at least one cutting component, wherein the retention bar supports the at least one cutting component.

8. The portable identification device of claim 1, wherein the body includes a top portion and a bottom portion, wherein the top portion is removably connected to the bottom portion.

9. The portable identification device of claim 1, wherein the body includes a slot to receive the identification card.

10. The portable identification device of claim 9, wherein the at least one cutting component is positioned to run along an end of the slot.

11. A portable identification device comprising:
a body;
at least one cutting component; and
at least one covering component;
wherein the body is operable to receive an identification card;
wherein the at least one cutting component and the at least one covering component are pivotably connected to the body;
wherein the at least one covering component is operable to distribute a layer of material over the identification card; and
wherein the at least one cutting component is designed to cut the layer of material and/or the identification card.

12. The portable identification device of claim 11, wherein the body includes a top portion and a bottom portion, wherein the top portion is removably connected to the bottom portion.

13. The portable identification device of claim 11, wherein the body includes a slot to receive the identification card.

14. The portable identification device of claim 13, wherein the at least one cutting component is positioned to run along an end of the slot.

15. The portable identification device of claim 11 further comprising an identification component, wherein the identification component includes a stamping tool, wherein the stamping tool is operable to stamp the identification card.

16. The portable identification device of claim 11 further including a dust container and/or a dusting wand.

17. The portable identification device of claim 11, including at least two grip components, wherein at least one grip component is positioned on a first end of the body, wherein at least one grip component is positioned on a second end of the body.

18. The portable identification device of claim 11 further comprising a pivot bar and a retention bar, wherein the pivot bar enables storage of the at least one cutting component, wherein the retention bar supports the at least one cutting component.

19. A portable identification device comprising:
a body;
at least one cutting assembly comprising a cutting component and a pivot component; and
at least one covering component;
wherein the body is operable to receive an identification card via at least one slot;
wherein the at least one cutting component and the at least one covering component are pivotably connected to the body;
wherein, via the pivot component, the at least one cutting component is operable to move between a stored position and an open position;
wherein the at least one covering component is operable to distribute a layer of material over the identification card; and
wherein the at least one cutting component is designed to cut the layer of material and/or the identification card.

20. The portable identification device of claim 19, wherein the at least one cutting component is operable to run along an end of the at least one slot.
